(19) United States
(12) Patent Application Publication
(10) Pub. No.: US 2017/0354383 A1
(43) Pub. Date: Dec. 14, 2017

(54) SYSTEM TO DETERMINE THE ACCURACY OF A MEDICAL SENSOR EVALUATION

(71) Applicants: Adam Odessky, San Francisco, CA (US); Ivana Schnur, Emerald Hills, CA (US); Ryan Connolly, San Francisco, CA (US); Cathy Pearl, Belmont, CA (US); Jeffrey Armbrecht, Menlo Park, CA (US)

(72) Inventors: Adam Odessky, San Francisco, CA (US); Ivana Schnur, Emerald Hills, CA (US); Ryan Connolly, San Francisco, CA (US); Cathy Pearl, Belmont, CA (US); Jeffrey Armbrecht, Menlo Park, CA (US)

(73) Assignee: Sense.ly Corporation

(21) Appl. No.: 15/177,220

(22) Filed: Jun. 8, 2016

(51) Int. Cl. A61B 5/00; A61B 5/16; A61B 5/145; H04L 29/08; A61B 5/0205; A61B 5/024; A61B 5/021; A61B 5/08

(52) U.S. Cl. CPC A61B 5/7289; A61B 5/7221; A61B 5/7435; H04L 67/12; A61B 5/165; A61B 5/0205; A61B 5/14532; A61B 5/4803; A61B 5/021; A61B 5/0816; A61B 5/024

(57) ABSTRACT

The present system discloses a data acquisition server for monitoring sensor data on a patient, the server controlling a plurality of sensors, each comprising a data acquisition circuit for measuring data on the patient, the server being operable to receive selection of a first sensor for capturing a first data on a patient, retrieve in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor, controlling the data acquisition circuit of the first sensor to capture first data, controlling the data acquisition circuit of each secondary sensors from the subset of sensors, to capture secondary data, retrieve in the sensor database a first rendering rule associated to the first sensor, the first rendering rule defining rules to process the second data prior to rendering, rendering on a user interface controlled by the data acquisition server the captured first data and the second data based on the first rendering rule.

### FIELD OF THE PRESENT SYSTEM

The present system relates generally to a technique for obtaining health information related to a user, and more specifically to a system for measuring the accuracy of medical sensor readings.

### BACKGROUND OF THE PRESENT SYSTEM

Medical sensors to measure physiological data on a user or patient benefits from the current communication revolution as such sensors may be used at home by the patient once connected to a local network. These sensors, just like any connected objects, may collect data that are shared remotely to a physician for instance. Medical monitoring systems have developed in the recent years to the benefit of a technical field referred to as telemedicine.

Such systems are for instance known from document U.S. Pat. No. 7,233,876B2, which describes a data acquisition system comprising a plurality of data acquisition sensors. A second sensor measures simultaneously with a first sensor the same critical parameter so that a backup solution is proposed.

This solution works for a controlled environment where no other parameters may alter both sensor readings. In a telemedicine system, various parameters or external conditions may affect the context and consequently the accuracy of a medical sensor reading on a patient.

Today there is still a need for a telemedicine system that provides contextual data about an ongoing medical sensor measurement so that the physician or practitioner may be able to interpret the accuracy of the results.

### SUMMARY OF THE PRESENT SYSTEM

The present system discloses a data acquisition server for monitoring sensor data on a patient, the server controlling a plurality of sensors, each comprising a data acquisition circuit for measuring data on the patient, the server being operable to:
receive selection of a first sensor for capturing a first data on a patient,
retrieve in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
controlling the data acquisition circuit of the first sensor to capture first data,
controlling the data acquisition circuit of each secondary sensors from the subset of sensors, to capture secondary data,
retrieve in the sensor database a first rendering rule associated to the first sensor, the first rendering rule defining rules to process the second data prior to rendering, rendering on a user interface controlled by the data acquisition server the captured first data and the second data based on the first rendering rule.
   each secondary sensor is also associated to an analysis rule that may depend or not upon the selected first sensor (defines frequency, processing of the raw data.

Such a system enables a practitioner to access not only a first parameter or physiological indication for a user, but also secondary or contextual parameters that describe the context of the capture of the first parameter. This may explain for instance abnormal values for a first parameter, like a state of nervousness that could alter values such as heartbeat, blood pressure, breathing cycles and the likes.

The present system also discloses method for monitoring sensor data on a patient using a data acquisition server, the server controlling a plurality of sensors, each comprising a data acquisition circuit for measuring data on the patient, the method comprising the acts of:
receiving selection of a first sensor for capturing a first data on a patient,
retrieving in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
controlling the data acquisition circuit of the first sensor to capture first data,
controlling the data acquisition circuit of each secondary sensors from the subset of sensors, to capture secondary data,
retrieving in the sensor database a first rendering rule associated to the first sensor, the first rendering rule defining rules to process the second data prior to rendering,
rendering on a user interface controlled by the data acquisition server the captured first data and the second data based on the first rendering rule.

The present system also discloses program product stored on a non-transitory computer-readable storage medium, and executable by a computer in the form of a software agent including at least one software module set up to implement a method for monitoring sensor data on a patient using a plurality of sensors, each comprising a data acquisition circuit for measuring data on the patient, the computer product comprising instructions to:
receive selection of a first sensor for capturing a first data on a patient,
retrieve in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
control the data acquisition circuit of the first sensor to capture first data,
control the data acquisition circuit of each secondary sensors from the subset of sensors, to capture secondary data,
retrieve in the sensor database a first rendering rule associated to the first sensor, the first rendering rule defining rules to process the second data prior to rendering,
render on a user interface controlled by the data acquisition server the captured first data and the second data based on the first rendering rule.

### BRIEF DESCRIPTION OF THE DRAWINGS

The present system is explained in further detail, and by way of example, with reference to the accompanying drawings wherein:

FIG. 1A shows a system in accordance with a first embodiment of the present system;

FIG. 1B shows a data monitoring platform in accordance with another embodiment of the present system;

FIG. 2 shows a flow diagram illustrating a process in accordance with embodiments of the present system;

FIGS. 3A-3F show exemplary interfaces to monitor a parameter for a patient in accordance to another embodiment of the present system;

FIG. 4A-4D show exemplary interfaces for a practitioner to configure the present telemedicine platform for a parameter in accordance with embodiments of the present system; and,

FIG. 4E-4F show exemplary interfaces to register a medical sensor for a user in accordance with embodiments of the present system.

### DETAILED DESCRIPTION OF THE PRESENT SYSTEM

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, etc. However, it will be apparent to those of ordinary skill in the art that other embodiments that depart from these details would still be understood to be within the scope of the appended claims.

Moreover, for the purpose of clarity, detailed descriptions of well-known devices, circuits, tools, techniques, and methods are omitted so as not to obscure the description of the present system. It should be expressly understood that the drawings are included for illustrative purposes and do not represent the scope of the present system. In the accompanying drawings, like reference numbers in different drawings may designate similar elements.

For purposes of simplifying a description of the present system, the term electronic device may refer to communication devices such as a personal computer (PC), a tablet (e.g., iPad™), a personal digital assistant (PDA), a mobile phone, a cellular phone, a smart phone (e.g., an iPhoneυ), a connected object, a medical device or sensor (e.g., blood glucose sensor, etc.), and/or other device for communicating using wired and/or wireless transmission methods.

The term rendering may refer to providing content, such as digital media which may include, for example, an electronic medical record (EMR), image information, information generated and/or accessed by the present system, messages, status information, settings, audio information, audiovisual information, sensor information, etc., such that it may be perceived by at least one user sense, such as a sense of sight and/or a sense of hearing. For example, the present system may render a graphical user interface (GUI) on a display device of an electronic device so that it may be seen and interacted with by a user. Further, the present system may render audio visual content on both of a device that renders audible output (e.g., a speaker, such as a loudspeaker) and a device that renders visual output (e.g., a display). The term content should be understood to include audio content and/or visual content (e.g., medical images), audio visual content (medical videos such as sonograms, etc.), textual content, and/or other content types, such a complex content comprising content of different nature such as an electronic medical record.

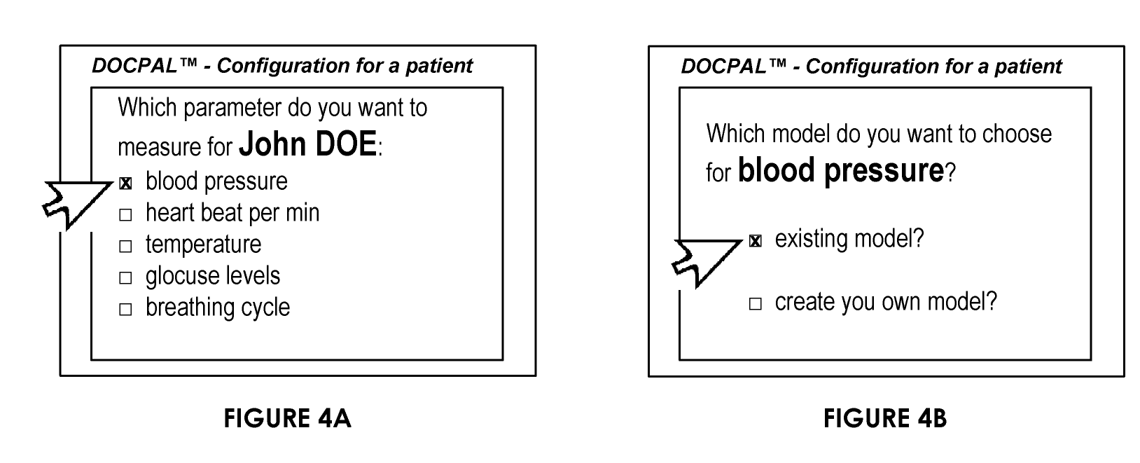

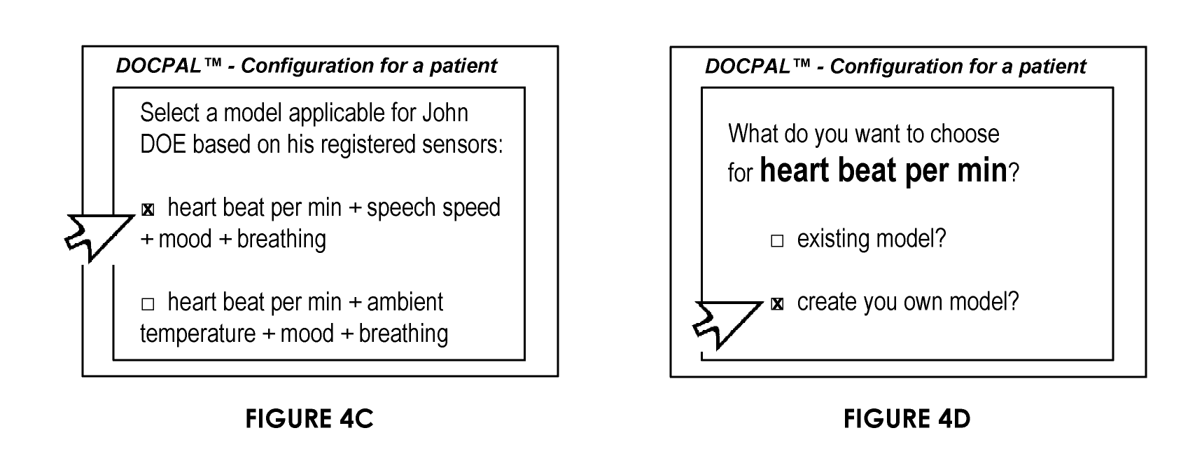

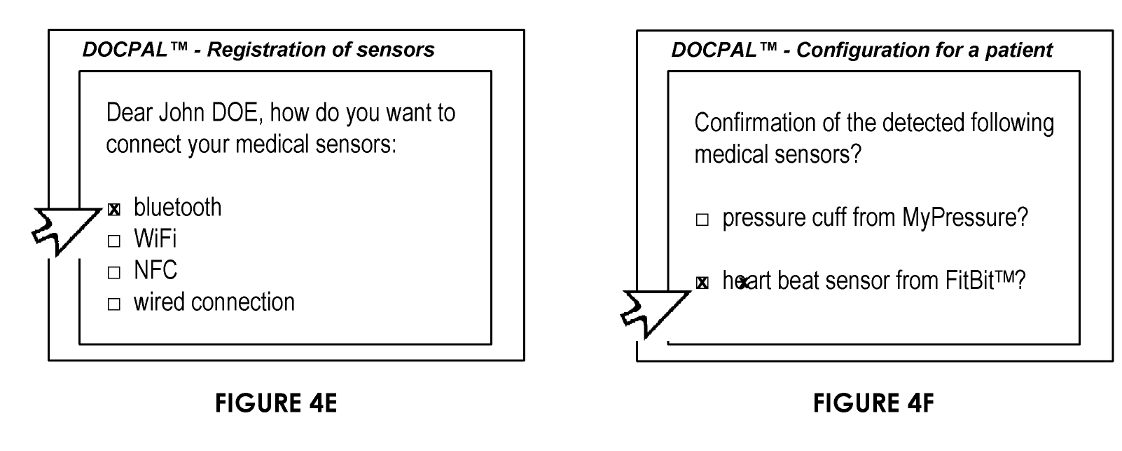

An example of a GUI in accordance with an embodiment of the present system is a GUI that may be provided by a computer program that may be invoked by the system or user, such as to enable a user to interact (e.g., provide commands, provide sensor data, etc.) with the system. FIGS. 4A-4D illustrate exemplary GUIs a practitioner may access when interacting with the present telemedicine platform, while FIGS. 4E-4F illustrate exemplary GUIs a patient may interact with when registering a new sensor in this EMR.

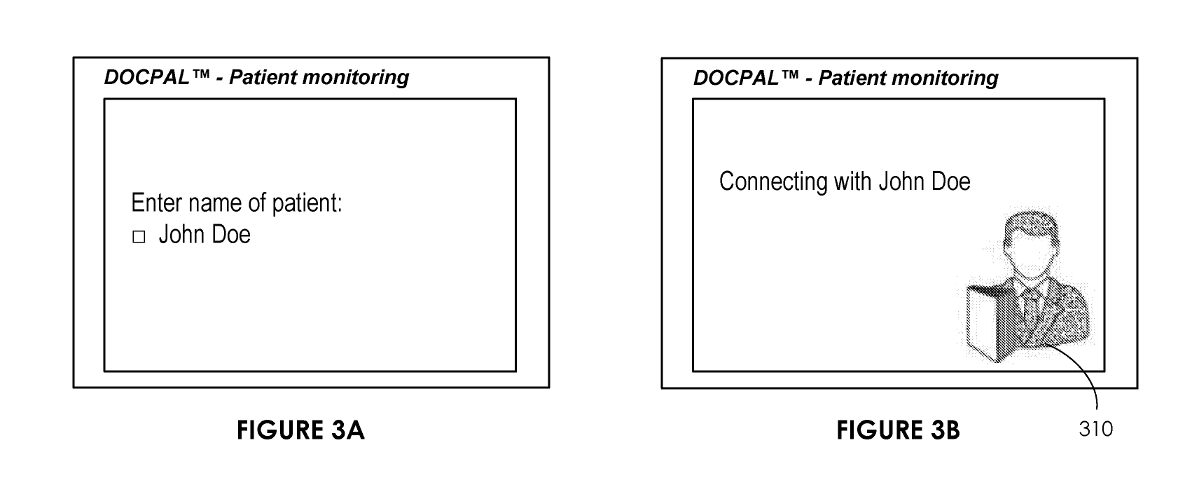

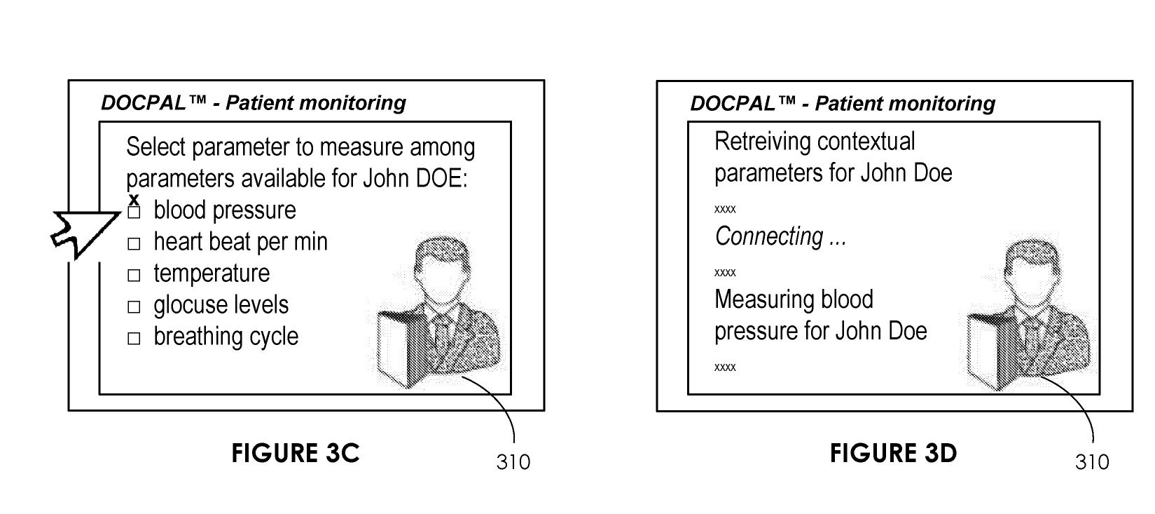

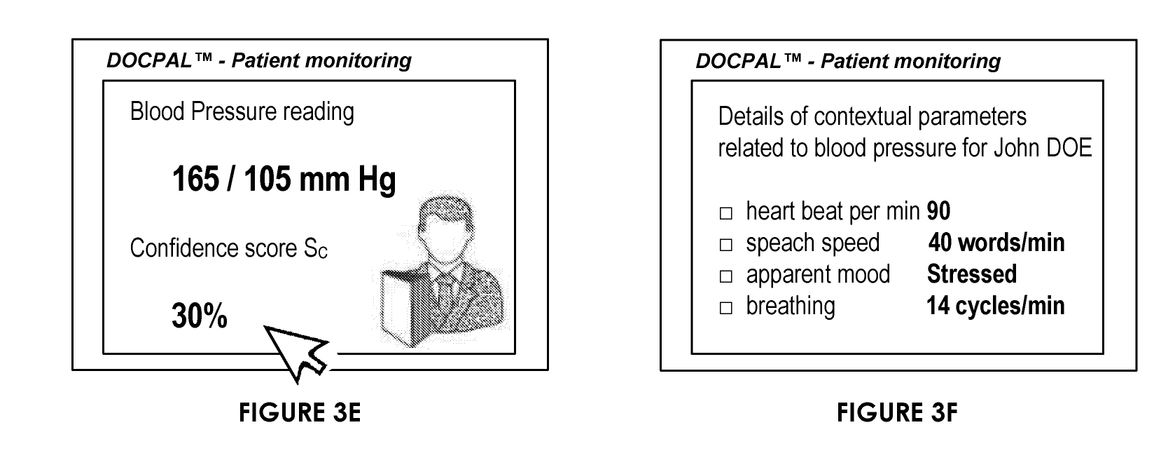

An EMR (electronic medical record), PHR (personal health record), PHR Extract and/or other type of medical summary, etc., is a computerized medical record which is organized (i.e., divided) per users (e.g., patients) and typically contains medical information (e.g., complete medical record) for each such user. The electronic record typically includes detailed information including clinical documents, lab reports, images (e.g., x-ray, CT scan, MRIs, etc.), trend data, monitoring data etc. Typically these records are created in an organization that delivers care, such as a hospital, doctors office, dentist office etc., although may also include records from non-medical facilities such as insurance offices etc. To simplify the following discussion, the term EMR will be utilized to include any such electronic data source that is organized and retrievable with regard to given users. In fact, EMR is actually made up of many individual records related to the patient. In the present system, the EMR may also comprise the different medical sensors registered with the patient corresponding to the EMR, medical sensors that may be controlled by the present telemedicine platform to perform a number of remote measurements of physiological indications. The EMR will further comprise the sensors characteristics such as made, model, identifier, address in the patient's network, and history of readings from the different captures triggered either by the practitioner or the patient himself. The EMR may further comprise the contact details or data for reaching the patient with the present telemedicine platform. This may be carried out through a video call with a video player added to the platform interface as detailed here after and seen from the exemplary user interfaces of FIGS. 3B-3G.

The problem with conventional telemedicine platforms is that they generally rely upon the measurement of one or more medical parameters individually. The doctor or practitioner is left with the interpretation and will have to take time in questioning the patient to better understand the context of the measurements. This may lead to a strenuous process both for the patient and the practitioner. The later may have to consult the complex medical record of the patient in order to better understand the context of the patient, and such information may not even be enough to reflect the state of the patient at the moment of the capture of the medical data.

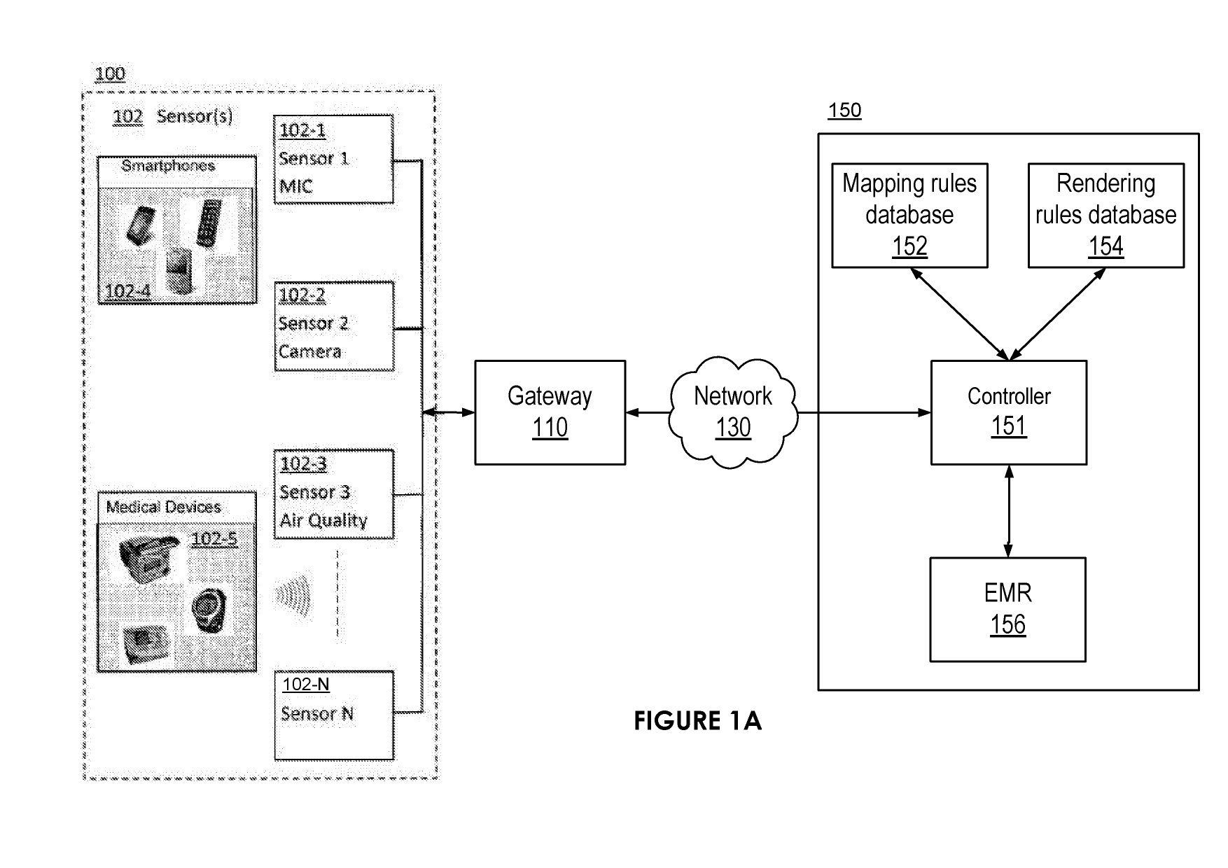

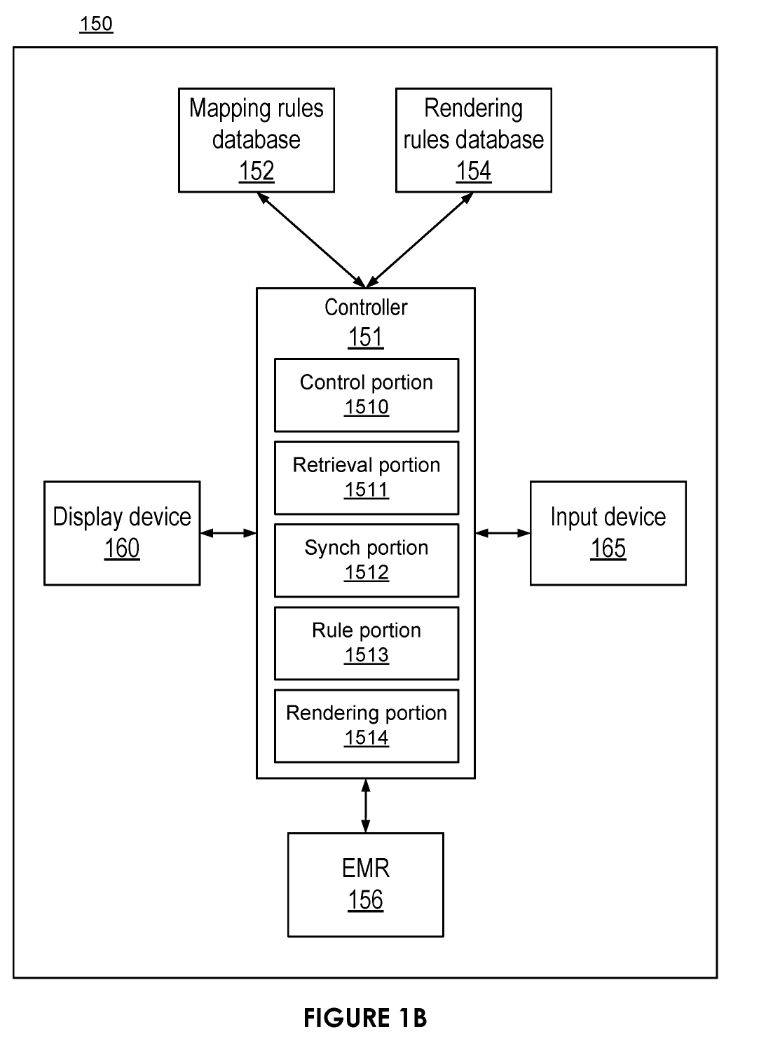

FIG. 1 shows a telemedicine system in accordance with an embodiment of the present system. The system may comprise a telemedicine platform 150 hosting a telemedicine solution, referred to here after as the DocPal service. The DocPal platform 150 may comprise a server or controller 151 providing access to a practitioner like a doctor at an office or practitioner venue to a plurality of telemedicine services. Among such services, the controller 151 enables the monitoring of a patient, e.g. at a home venue, through a patient side system 100 comprising a plurality of connected sensor devices 102.

The sensors 102 may include first through N^{th} sensors (or groups of sensors) such as sensor 102-1 (sensor 1) through 102-N (generally 102-x) which may obtain information related to, for example, physiological indications of a user, environmental conditions (e.g., temperature around the patient, barometric pressure, pollen count, etc.), locations (e.g., of the user), images (e.g., image information), etc. More generally the sensors will be referred to as measuring one or more parameters or indications relative to the patient. Each sensor 102-x in the present system may comprise a data acquisition circuit that may receive acquisition signals from one or more controllers to capture the physiological indications, or more generally data (for instance images, sounds . . . ) that may be further analyze either locally or remotely to deliver the physiological indications.

In accordance with embodiments of the present system, physiological indications may include information related to physiologic parameters of a user such as pulse, breathing (e.g., information indicative of breaths per minute (BPM), duration of breath cycles (e.g., inhale, exhale, etc.)), temperature, blood glucose level, physiological pressures (e.g., blood pressure), electrical activity (e.g., EKGs, etc.), chemical levels (e.g., O₂, N₂, CO, etc.), biologic levels (e.g., white or red blood cell count, etc.), presence of pathogens, and/or other information which may be indicative of biological states, functions, levels, activities, etc., of a user and may be sensed by one or more of the corresponding sensors 102-x, such as medical sensors 102-5. A blood pressure cuff and a halter-monitor (or heartbeat monitor) are examples of such medical sensors.

In accordance with embodiments of the present system, environmental conditions (e.g., air quality) may include temperature, humidity, barometric pressure, O₂/CO₂/NO₂ levels, chemical and/or biological compounds, contaminants (e.g., pollen, pollutants, dust, etc.), radiation levels, particulate levels, wind speed, ultraviolet (UV) radiation, infrared (IR) radiation levels, etc., and may be sensed by one or more of the corresponding sensors 102-x, such as an air quality sensor 102-3.

Image information may include, for example, still images, video images (e.g., a sequence of images of a user), medical images (e.g., magnetic resonance imaging (MRI) images, X-ray images, computed tomography (CT) scans, etc.), lighting conditions (e.g., light levels, etc.), and may be obtained using one or more suitable sensors 102-x. The sensors 102-x may continually output (e.g., by pushing to the system, etc.) sensor information and/or may output sensor information in response to received signals such as a control signal (CON) from, for example, the controller 151. The sensor information from each sensor 102-x may include meta information related to its context such as an identity of the sensor, a type of sensor information (e.g., sound information, video information, blood glucose level, air quality, etc.), time (e.g., 2:00 am, Jul. 31, 1998, etc.), location (e.g., geophysical location, network address location, etc.), user ID, etc. The sensor information may include processed and/or raw information in analog and/or digital form. Further, one or more of the sensors 102-x may be remotely located from the user (e.g., a web-based air quality sensor, etc.).

More generally, in accordance with embodiments of the present system, a device 102-x, referred to as the first or primary sensor, may be selected by the practitioner through the telemedicine platform 150 to monitor a first parameter and capture first data value on the patient. Thanks to a first mapping rule associated to the first sensor, a plurality or subset of secondary sensors 102-y may be operated by the DocPal platform 150 to capture secondary data values along with the first data values. The secondary data (values) captured by the secondary sensors will be used in the present system as contextual data to help the practitioner better understand the first parameter data thanks to the present DocPal telemedicine service.

Some sensors may be dedicated sensors, like a medical sensor such as a blood pressure cuff, a heartbeat monitor or a temperature gage. Additionally, some sensors may be hosted on a same device like a smartphone 102-4, which may comprise a camera and a microphone. All sensors as described here after may be registered for their different capacities (i.e. each parameter they may capture), so that each capacity may be addressed individually by the controller 151.

Further, with respect to the sensors 102-x, one or more of the sensors 102-x may provide different types of information. For example, with regard to various physiological indicators of the user (e.g., pulse, breaths per minute, mood etc.), associated information may be captured by different sensors and processed in accordance with the type of sensor capturing the sensor information. For example, a user's pulse rate may be determined by processing an image of a user's artery or vein captured by a camera of the smartphone 102-4 or may be determined directly by a medical device such as the camera sensor 102-2. Similarly, breaths per minute (BPM) of a user may be determined by analyzing suitable information such as audio information of a user breathing which may be captured by a microphone of, for example, the same smart phone 102-4 or directly by a dedicated microphone 102-1. Similarly, a mood of a user may be measured through the analysis of a video captured by a camera sensor, using for instance mood analysis software like Affectiva™ which allows to classify a user's face according to predefined moods, like happy, sad, stressed, fear, disgust, surprise, contempt . . . Other software may be used to analyze the user's speech, either speedwise (fast or slow phrases) or through its tone (emotional, sensitive, stressed . . . ). A speech to text may also be used to analyze what the user may be conveying to the practitioner during the consultation.

More generally, some sensors may be considered as dedicated medical sensors as they can deliver directly a physiological indication for the patient. Other sensors may be considered as non-dedicated medical sensor as the captured data will require some processing in order to derive an indication useful to the practitioner.

The sensors 102-x may form a part of a network such as, for example, a personal area network (PAN) of the patient, a local area network (LAN), a wide area network (WAN), a distributed network, a peer-to-peer (P2P) network, and/or may communicate with each other and/or other portions of the patient side system 100 using any suitable communication method such as a wired and/or wireless communication method (e.g., the Internet, Bluetooth™, etc.).

The sensors 102-x may be stand-alone sensors (e.g., a remote temperature sensor such as a web-based municipal air-quality sensor, a Bluetooth™ enabled MIC, etc.), mobile station sensors (e.g., smart phone sensors such as camera, MIC, temperature sensor, location sensor, etc.), and/or medical equipment sensors (e.g., EKG sensors, etc.).

The patient side system may be scalable by adding more sensors 102-x to the existing sensors. Communication with the telemedicine platform 150 may be ensured through a communication network 130 accessed through a gateway 110 like a router to connect the patient's premises to the Internet. The gateway may be one of the smart phones or mobile devices 102-4 mentioned before. In the present system, for any registered patient with the telemedicine platform 150, a list of available sensors may be stored with an entry for the patient in his electronic medical record (EMR) 156. For instance, each sensor may be addressable through a unique identifier and/or an IP address saved in the EMR 156 in the patient's record. The present controller 151 operating the telemedicine platform 150 is configured to drive such sensors, through control signals, when a capture or sampling for sensor data is requested by the practitioner.

The present telemedicine process may be performed using one or more computers or controller 151 communicating over a network 130. The process may include one of more of the following portions which may perform one or more acts, sub-acts, etc., desired. One or more of these portions may be implemented as a program executed by a processor of the controller 151 thereby causing the processor to execute instructions for performing the method in accordance with the present system.

The EMR may be stored in a medical information memory (MIM) 156 of the present telemedicine platform 150. The MIM 156 may be any suitable memory such as a local memory, a remote memory, and/or distributed memory (e.g., a surface area network (SAN), etc.).

In the present system, for each entry for a user in the MIM or EMR database 156, the list of registered or connected medical sensors 102-x is stored. The list will also comprise for each sensor:
an identifier, a network location address, and metadata to describe its capacities, such as made and model. The network location address may be used in the present system by the controller 151 to address control and data acquisition signals to the sensors so as the trigger the capture of data,
the physiological indication(s) or parameter(s) that can be captured by the sensor, and what processing, if any, is needed to transform the captured data into physiological parameter(s).

A sensor may indeed capture one or more parameters. For instance a blood pressure cuff may measure both the blood pressure of a patient and his heartbeat per min. Generally speaking, in the description hereafter, a physiological and/or contextual parameter and the sensor capturing it may be used indifferently. For instance, if the practitioner selects a parameter P to capture, the corresponding sensor 102-P will be automatically selected for capturing the parameter values for the patient. In the even several sensors can capture the same parameter, like the heartbeat per min that may be captured by either a halter monitor, a blood pressure cuff or a camera (see before), the practitioner may be offered to choose one of them. Alternatively, the telemedicine platform 150 will select one by default or based on availabilities at the patient venue.

A mapping rule or sensor database 152 is further accessible to the controller 151 in the telemedicine platform 150. A mapping rule defines for a first parameter a list of associated or linked secondary parameters that will provide to the practitioner valuable information to interpret and understand the context of the first parameter values as reported by the first sensor. Consequently, the mapping rule will also associate for a first sensor 102-P available to capture the first parameter a subset of secondary sensors 102-y among a plurality of sensors 102-x available to the telemedicine platform for capturing contextual data.

Any parameters monitored with the present telemedicine platform 150 may be associated in an entry of the mapping rule database 152 to one or more mapping rules, linking it to other parameters called contextual parameters. The mapping rule may be predefined, i.e. corresponding to a predefined model as set by the system, and originating for instance from well-known models generally accepted in the medical field. Such predefined model will associate a first parameter to secondary parameters known as being particularly impacting on the first parameter values. Say the first parameter is the blood pressure. A predefined model may be for instance a model linking the blood pressure to the heartbeat per min, the speech speed, the mood and the breathing rhythm, as seen in the illustration of FIG. 4C. The predefined model may be used across any patients. It may nonetheless be altered if a patient under care does not have some of the sensors needed for the secondary parameters from the predefined model.

Looking at FIG. 4C, the practitioner may choose an existing or preset mapping rule for blood pressure that associates this physiological indication to the following secondary parameters:
heart beat per minute,
speech speed,
mood,
breathing,
temperature,
sugar levels.

The proposed interface of FIG. 4C shows that the mapping rule may be adapted to the existing (i.e. registered in his EMR) sensors for the patient under review. For this patient, the temperature and sugar level sensors are not available and the selected mapping rule will be limited to the heart beat per minute, the speech speed, the mood and the breathing. The other secondary parameters (temperature and sugar levels) are discarded as not measurable due to the absence of the corresponding sensors.

The mapping rule for a parameter may be customized by the practitioner if he is more interested in using specific secondary parameters that he considers as more impacting on the measurement of a primary parameter. This is illustrated for instance in FIG. 4D wherein the practitioner can create his own model for the first parameter heartbeat per min.

When several mapping rules are available for a first parameter, the telemedicine platform 150 may be arranged to find the most suitable mapping rule for a patient based on the sensors 102-x registered in his EMR entry in the EMR database 158. A most suitable mapping rule for a patient may be for instance the one requiring the largest number of sensors registered for that patient. Alternatively some secondary parameters may be weighted differently in a mapping rule. The most suitable mapping rule for a patient may be the one where the parameters with the highest weights are measurable for that patient.

A mapping rule may define for the first parameter and each secondary parameter configuration settings like time of measurement, duration, software to analyze the captured data from the sensor . . . so that the secondary parameter may be captured simultaneously with the first parameter. The configurations settings of a sensor are used by the controller 151 to generate the signals to control the data acquisition circuit of the sensor in order to capture data. Some data from a sensor may be considered as “raw data” that needs further processing for obtaining the needed contextual or physiological data. Indeed, when the blood pressure is selected as the first or primary parameter to monitor on a patient, the mapping rule pointing to the heartbeat per minute as one of the secondary parameter may further specify for that secondary parameter a starting point and a duration for the measurement, as well as other settings. if the heartbeat per minute is also measured using the blood pressure cuff, the settings may be set to measuring the heartbeat per minute during the same time that is necessary to capture the value for the primary parameter “blood pressure”.

In the present telemedicine platform 150, a rendering rule database 154 is further accessible for displaying the secondary parameters measured with the first parameter. To each mapping rule for a first parameter may correspond a rendering rule for handling the secondary parameters measured simultaneously (with that first parameter) in view of their rendering. In other words, a rendering rule defines rules to process the secondary data prior to its rendering. The rendering rule may define for instance a confidence score resulting from a combination of values measured for the secondary parameters.

Going back to the example of FIG. 4C, the rendering rule may define different thresholds for each secondary parameter as follows:
heart beat per minute:

| heartbeat per | |
|---|---|
| minute | score |
| <75 | 0 |
| >75 | 1 |
| >90 | 2 |

speech speed:

| words per | |
|---|---|
| minute | score |
| <40 | 0 |
| >40 | 1 |
| >55 | 2 |

mood: as mentioned before, mood may be measured using a software like Affectiva™,

| mood as analyzed | |
|---|---|
| by Affectiva ™ | score |
| stressed | 3 |
| sad | 1 |
| happy | −1 |
| fear | 2 |

breathing: if number of breathing cycles per min is greater than 15, score 1,

| breathing cycles | |
|---|---|
| per minute | score |
| <12 | 0 |
| >12 | 1 |
| >16 | 2 |

The scores from the rendering rules may be summed up over the different monitored secondary parameters. In the present example, the resulting score may even be normalized so as to give a score, referred to here after as a reliability score S_{R}, over 100. In this illustrative embodiment, the higher the reliability score is, the less reliable the measurement of the first parameter may be. Such a score will give an idea to the practitioner that the blood pressure value currently measured for the patient, if high, could be heavily influenced by the current nervous state of the patient. In the illustration of FIG. 3E, corresponding to the same mapping rule as in FIG. 4C, the score would be 7 based on the previous tables, for a maximum score of 10, giving a reliability score of 70%, or a confidence score S_{C}=1−S_{R}=30%. In the present system, the practitioner may even select the confidence score to get a breakdown of the monitored secondary parameters to get a detailed view on the factors contributing to the high blood pressure reading of FIG. 3E.

Alternatively, the confidence score may be rendered through discreet values so as to qualify the risk associated to the measurement of the primary parameter with a threshold approach. The risk associated to the measurement of the primary parameter may be stratified as:
High Risk with S_{C}<33%, as the measurement is unreliable, as heavily influenced by the contextual paramaters,
Medium Risk with 33%<S_{C}<67%, as the measurement is relatively influenced by the secondary parameters,
Low Risk with S_{C}>67%, as the measurement is fairly reliable.

FIG. 1B shows a more detailed embodiment of a telemedicine platform 150 in the present system. The platform 150 may further comprise an input device 165 and a display device 160 so that the practitioner may interact with the controller 151 that will execute embodiments of the present method. This may include for instance:
the identification of a patient,
the selection of a first parameter,
the retrieval of the associated to secondary parameters defined in the mapping rule of the first parameter (as illustrated in the FIGS. 3A-3F),
the configuration of the present system as illustrated in the different interfaces shown in FIGS. 4A-4D, through the selection or definition of mappings rules for different first parameters and associated sensors,
the registration of sensors on the patient side as shown in FIGS. 4E-4F. . .

The controller or processor 151 of the present telemedicine platform 150 may comprise different portions or units addressing different tasks for the platform. The controller 151 may include a controller portion 1510, a retrieval portion 1511, a synchronizer portion 1512 (synch portion here after), a rule portion 1513 and a rendering portion 1514.

Further, operative acts of portions 1510 through 1514 may be performed using software (e.g., using applications) and/or hardware. The software may be stored in a memory of the telemedicine platform 150. For example, the control portion 1510 may be part of the controller 151 or server 150, and may include one or more processors, logic devices, etc., to control and execute the overall operation of the telemedicine platform 150.

The control portion 1510 is arranged to control the different interfaces to interact either with the practitioner on the practitioner side (e.g. at his office) of the telemedicine platform 150 or the patient on the patient side of the platform (e.g. at his home). On the practitioner side, this may involve the selection of a patient (see FIG. 3A), the selection of a primary parameter or sensor (see FIG. 3C), the provision of the sensors output (see FIG. 3E) or details of the output (see FIG. 3F). This may also involve interfaces to interact with the EMR of the patient, connect and exchange with the patient (see FIG. 3B), or configure the different mapping rules (see FIGS. 4A-4D). On the patient side, this may involve interfaces for the patient to access his EMR or manage and/or register new sensors (see FIGS. 4E-4F).

The retrieval portion 1511 is arranged to access and retrieve the different user and sensor information that may be needed by the control portion 1510 to enable the present method. For instant, the control portion 1510, upon selection of a patient through an interface like the exemplary interface of FIG. 3A, may drive the retrieval portion 1511 to access the EMR and retrieve the patient's EMR. The EMR, as mentioned before may comprise the patient's contact details as well as the different sensors and related parameters that may be monitored and captured for the selected patient.

Indeed, using the contact information for the patient retrieved by the retrieval portion 1511 in the EMR 156, the telemedicine platform 150, more specifically the control portion 1510, may set up a call or a video conference between the patient and the practitioner as seen in FIG. 3B. Through e.g. a video call plugin, a video player 310 appears on the practitioner side interface, once the patient responds to the call from the telemedicine platform.

Using the retrieved sensor information, the control portion 1510 is further arranged, as seen in the exemplary interface of FIG. 3C to display the different parameters that may be selected for the patient under care.

Once a first parameter, or a related first sensor is selected, the blood pressure in the exemplary illustration of FIG. 3C, the control portion 1510 will further drive the retrieval portion 1511 to select in the mapping rule database 152 the mapping rule associated to the selected first parameter entry, presently the “blood pressure” entry. As mentioned before, the mapping rule may be a generic mapping rule indexed for the selected first parameter. Alternatively, it may be a user based mapping rule, the retrieval portion 1511 providing both the first parameter and a user identifier to select the relevant mapping rule. When a generic mapping rule is selected, that comprises secondary sensors or parameters that may not be available at the patient premises, the control portion may output a message to the practitioner mentioning the missing sensors. A prompt from on the interface may ask the practitioner if he wants to carry on besides the lack of some information. The control portion 1510 may comprise intelligence to dynamically adapt the mapping rule to the registered sensors for the patient, by discarding the other secondary parameters that are not measurable. The rendering rule may be adapted accordingly to the sole secondary parameters that can be measured for the patient under care.

In an additional embodiment of the present telemedicine platform, the control portion may comprise further intelligence to dynamically adapt a mapping rule to the current condition, sickness or ailment of a user, as stored in his EMR. For instance, if the patient is known to be stressed out whenever he is interacting with the practitioner, the system may adapt the mapping rule so as to capture more sensor data and physiological indications highlighting the level of stress of the user. More precisely if recent consultations have shown the patient to be more calm, the mapping rule may be adapted to include only a few basic secondary parameters. If the patient has shown himself to be more nervous in recent consultations, the mapping rule may be “reinforced” with further secondary parameters, like mood, speech speed, movements of the body, leading for instance to a more resource demanding processing of the image data captured the camera sensor 102-2.

Once the mapping rule is retrieved, the control portion 1511, aware of which sensors to monitor, will output acquisition signals to inform/control the synch portion 1512 to activate and acquire data from the corresponding sensors data using the sensor network address location as known from the patient's EMR. The information may be collected following the practitioner's request, or at periodic, continuously (e.g., real-time) and/or non-periodic intervals, depending on the type of sensors. Thus, when a breathing parameter is selected by the practitioner, the control portion 1150 may signal the synch portion 1512 to acquire sensor information from the microphone 102-1 over a two minute interval. Say the mapping rule links the breathing parameter to blood pressure, heartbeat, and mood, the control portion 1050 may activate and/or request sensor information from various sensors such as the camera sensor 102-2 for the mood and a medical device 102-5 like a blood pressure cuff for the blood pressure and heartbeat. The cuff may capture the blood pressure and the heartbeat twice over the 2 minutes so as to get an average value. The camera sensor 102-2 may capture images from the patient over a minute, so as to send the captured data to a remote server running a software like Affectiva™. A shorter capture time for the camera will allow time for the mood analysis to be ready when the capture of the microphone 102-1 and the blood pressure cuff 102-5 are done after 2 minutes.

The synch portion 1512 may aggregate the sensor information which it receives from the plurality of sensors 102-x and output this information to the control portion 1510 for further processing. Such output sensor information will include the primary sensor data and further contextual sensor information from the associated secondary sensors.

The control portion 1510, once the sensor information (hereinafter received sensor information) is received from the synch portion 1512, may further process it to analyze types and/or values of the received sensor information (e.g., temperature, air quality, blood glucose level, image information, audio information, location, etc.) and/or perform acts in accordance with a context of the received sensor information (e.g., perform blood glucose test, breathing analysis, etc.). For example, the control portion 1510 may process the received sensor information to determine various medical conditions (e.g., diabetes, apnea, stress levels, allergies, etc.). For example, in accordance with embodiments of the present system, the control portion may include intelligence to analyze and predict possible medical hazards to the user based on the sensor information.

Further, if it is determined that the sensor information includes specific type of data that may require further processing such as audio and/or image information (e.g., an analysis with the Affectiva™ software, voice record analysis for determining breathing rhythm, speech speed, word analysis for mood etc.), the control portion 1510 may transmit such sensor information to other optional portions where the specific type of data may be processed. Such portions (not shown in FIG. 1B) may then return the results of the further processing to the control portion 1510. The received sensor information, together with the further processed sensor information will form the output sensor information that is ready for the subsequent rendering to the practitioner in the present telemedicine platform 150.

Generally speaking some sensor data may be processed locally by the sensor itself so as to produce sensor information that does not need any additional processing by the control portion 1510 to produce the physiological or contextual information. This is for instance the case of the blood pressure cuff for the blood pressure or heartbeat per minute parameters. Alternatively, some sensor data may need further processing once captured, like image or voice analysis in order to produce the physiological or contextual information. The further processing may be executed prior the retrieval by the synch portion 1512, either at the patient's premises on one of his electronic devices or remotely through cloud computing. The further processing may be done as mentioned before by the other optional portions mentioned just before.

In order to prepare the rendered sensor information as shown in the illustration of FIG. 3E, the control portion 1510, once all the output sensor information is collected, will request from the retrieval portion 1511 the rendering rule associated in the rendering rule database 154 to the selected primary parameter. As mentioned before, different type of rendering rule may be defined for a first parameter. The rendering rule may be a generic rendering rule, corresponding to a generic mapping rule for the first parameter. It may be a customized rendering rule, e.g. tailored by the practitioner or mapped to the patient's registered sensors, to adapt it to the patient. A rendering rule may be advantageously paired to a mapping rule for a first parameter so that all captured sensor data are taken into account in the rendering. Alternatively, the control portion 1510 may comprise intelligence to adapt the chosen rendering rule to the mapping rule so as to avoid any system errors. Once retrieved, the rendering rule is applied by the control portion 1510 to the output sensor information (including the information that required further processing).

The control portion 1510 is further operative to update the patient's EMR with the different sensor information in order to the keep the patient's record up to date.

A mapping rule portion 1513 and a rendering rule portion 1514 are further available in the present controller 151 to manage and update the mapping rule database 152 and the rendering rule database 154 respectively as described later on.

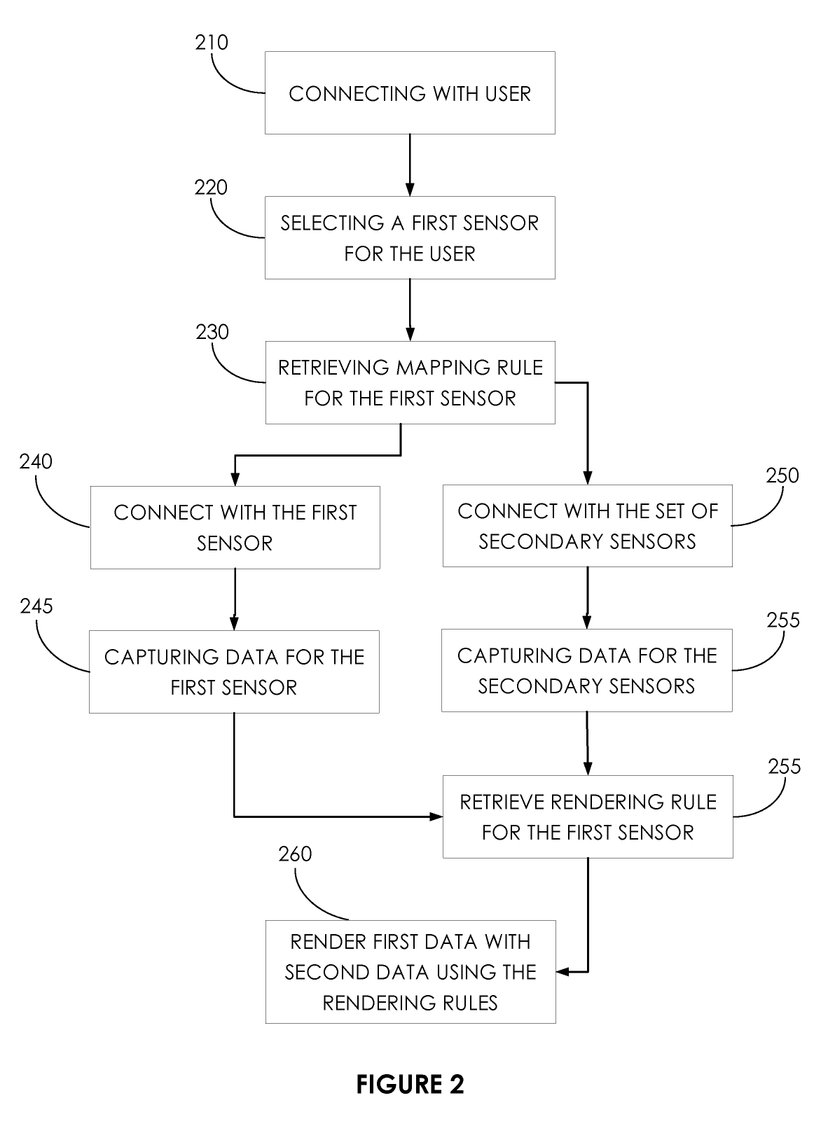

FIG. 2 shows a flow diagram that illustrates a process 200 in accordance with an embodiment of the present system. The flow diagram of FIG. 2 will be described hereafter in relation to the practitioner side user interfaces shown in FIGS. 3A-3F, displaying how a consultation may be carried out with the present telemedicine platform. The process 200 may be performed using one or more computers communicating over a network 130, like the server 151, comprising the different portions described herebefore.

The process 200 may include one of more of the following acts. In operation, the process may start with the practitioner identifying himself with the telemedicine service/platform using a secure login procedure beyond the scope of the present system. Once connected, the practitioner may select a patient first “John Doe”, referred herein as the patient under care as seen in the exemplary interface of FIG. 3A, showing a patient monitoring interface. The control portion 1510 will requested the retrieval portion 1511 to look into the EMR database 156 to retrieve John Doe's EMR, including his contact information. The process will move to a further act 210 of contacting the patient. This is shown in the illustration of FIG. 3B, wherein a video element 310 appears on the practitioner side interface, displaying a live video stream of John Doe, as captured for instance by a camera sensor 102-2 at the patient's premises.

The EMR will also include the plurality of sensors registered for John Doe, and related parameters that may be captured thank to the present telemedicine platform 150. Based on the list of measurable parameters, the control portion will form an interface as illustrated with the interface of FIG. 3C, displaying a list selectable elements, each corresponding to a parameter to monitor for John Doe, so that the practitioner may select at least one for review. In a further act 220, as seen in the same illustration of FIG. 3C, the practitioner will select a first parameter, here the blood pressure, to be measured during the consultation with the patient John Doe. The selection of one or more first parameters, referred to as the primary parameter(s), will cause the control portion 1510 to retrieve a corresponding (first) mapping rule indexed in the mapping rule database 152 in an entry for the first parameter (or first sensor). The control portion 1510 is now aware of the first sensor, along with a subset of secondary sensors among the sensors registered for the patient John Doe, that are linked through the mapping rule to the first sensor as they measure a type of data or parameters that characterizes the context of measurements by the first sensor. The control portion is also aware of the sensor identifier and network address location.

The retrieval of the mapping rule is illustrated in the practitioner side interface of FIG. 3D, showing a message to the practitioner that the contextual parameters for John Doe are being retrieved. In further acts 240 and 250, the control portion 1510 will connect with the first sensor and the subset of secondary sensors respectively, using the synch portion 1512 and the network address locations. This will be carried out by the synch portion 1512 checking if the sensors are online and e.g. sending a wake up instruction to their respective data acquisition circuits. As mentioned before, the mapping rule may further comprise configuration settings to define how the primary and secondary sensors may be controlled to capture the sensor information (or sensor data). The configuration settings for the secondary sensors may take into account the specificities of the primary sensor, for instance running time of the measurement, number of capture, type of output, pointer to a software or control portion if further processing is needed . . . so as to be synched during the capture.

The exemplary illustration of FIG. 3D will show the message “connecting” to inform the practitioner of the current advancement of the present process. The video element 310 showing the live stream of the patient may still be visible so that the practitioner may interact with the patient while the sensor measurements are carried out.

If a sensor is not available, i.e. the sensor is not responding to the wake up instruction from the synch portion 1512, a warning message may be displayed by the control portion 1510 to the practitioner so that the practitioner may interact either with the telemedicine platform 150 or the patient himself to check the status of the unreachable sensor.

In respective further acts 245 and 255, the sensor data for the first sensor and linked secondary sensors may be captured based on the respective configuration settings. Indeed, each sensor is further controllable through its data acquisition circuit mentioned before so as to capture sensor data. This will also be carried out through further acquisition signals received from the synch portion 1512, taking into account the sensor configuration settings. Another message “measuring blood pressure for John Doe” is displayed on the practitioner side interface of FIG. 3D. In response to the acquisition signals sent out to the (data acquisition circuit of the) different primary and secondary sensors, the telemedicine platform 150 will collect respective first sensor data and secondary sensor data.

Some of the received sensor data or information (the raw data mentioned earlier) may need further processed so as to form physiological parameters useful for the practitioner.

In parallel, or once output sensor data is collected, the control portion 1510 will retrieve the rendering rule for the selected first parameter using the retrieval portion 1511 in a further act 255. Applying the retrieved rendering rule to the collected secondary data, the control portion in a further act 260 will render the first data from the first sensor along with the secondary data processed according to the rendering rule on the display device 160 of the telemedicine platform 150. This is illustrated in exemplary interface 3E wherein the blood pressure results for John Doe are displayed with the processed secondary data, here shown in the form of a confidence score of 30%, giving the practitioner additional information that contextual parameters may have influenced the high blood pressure readings for John Doe. The confidence factor may be a selectable element on the graphical user interface of FIG. 3E so that the practitioner can zoom into the details of how the confidence score was built. In a further act of the present process, the practitioner may select the score S_{C} to have access to the details of the secondary parameters from the mapping rule. This is further shown in FIG. 3F showing that the mapping rule includes for the blood pressure as the first parameter:
heart beat per minute,
speech speed,
(apparent) mood,
breathing,

Values for these secondary parameters are also shown, all showing here that the patient John Doe may be under a relatively high level of stress, explaining his unusually high blood pressure. The illustrated score of 30% is based on the exemplary tables mentioned before for the rendering tules, leading the practitioner to question the validity of the blood pressure test. He may opt to carry another one later on the consultation whenever the patient John Doe becomes more relaxed.

In an additional embodiment of the present system, the rendering rule may include rules to combine the first sensor data with the linked secondary data. The interface would how a real blood pressure value and a corrected blood pressure value based on the contextual data. In the present illustration the actual blood pressure value would be 165/105 mm HG while the corrected value would be 130/90 mm HG, reassuring the practitioner that the actual value does not represent a warning to act upon.

As mentioned earlier, the practitioner may configure the present telemedicine service, notably through selecting different mapping and rendering rules. This may for instance be achieved through the practitioner side interface of the present telemedicine system illustrated in the exemplary embodiment of FIGS. 4A-4D. The GUIs are illustrations of a predefined mapping rule adapted to a specific patient John Doe. Alternatively the mapping rule may be a predefined or generated (by the practitioner) one to be used across any patient. The mapping rule may even be defined patient by patient, based on the sensors registered on their respective EMR.

When entering the “patient configuration” option in the present telemedicine platform as seen in FIG. 4A, for a selected patient John Doe, the user may see which primary parameters (and related sensors) are available for this patient. Provided he select e.g. blood pressure, the controller 1510 will prompt another interface as in FIG. 4B asking the practitioner whether he may want to use a predefined mapping rule, for instance defined by a panel of experts and made available to any practitioner when using the present telemedicine platform. The GUI in FIG. 4B offers the possibility as well to select the creation of the practitioner's own mapping rule for the patient John Doe. Following the selection of using a predefined model, the controller 1510 will retrieve among the predefined generic mapping rules one or more rules suitable for the current patient, for instance based on the sensors registered in his EMR. This is illustrated with the choice of either:
heart beat per min+speech speed+mood+breathing
heart beat per min+ambient temperature+mood+breathing

By filtering a predefined mapping rule with the registered sensors for John Doe, the practitioner can get a better idea of the most suitable secondary parameters to understand the first parameter values. In the present illustration of FIG. 4C, other secondary parameters may be used in the different mapping rules stored in the mapping rule database 152, but are hidden to the practitioner as not registered and consequently not available to be measured, with e.g. the blood pressure of John Doe. Returning to the GUI to select a mapping rule, the practitioner may opt as seen in FIG. 4D to create his own mapping rule as he is for instance aware of some specific parameters for the patient that are especially useful to measure and help with the understanding of each blood pressure check he may want for his patient.

FIGS. 4E-4F illustrate another embodiment of a patient side function available with the present telemedicine platform 150, namely the sensor registration function. Medical sensors may be connected to the platform and registered for a patient using different communication technologies or channels specific to the sensor. Several communication channels may be even used to connect a sensor. In order to access the registration option, the patient from his home venue will connect to the telemedicine platform using an electronic device configured to interact with the platform 150. When navigating through the menu after identification, and selecting the device registration option, a patient side interface as illustrated in FIG. 4E will appear on his electronic device, listing the different communication channels available for registering a new sensor. As he just acquired a heartbeat monitor that requires Bluetooth to communicate, he may select the Bluetooth™ channel. The electronic device will then explore available Bluetooth device, listing a pressure cuff and a heartbeat monitor as in the exemplary interface of FIG. 4F. The patient may then select one of the listed devices for registration with the telemedicine platform 150. A subsequent update to the patient EMR will allow the addition of the new sensor, e.g. its characteristics, parameters to be measured, driver to control the sensor, configuration settings, further processing if needed, network address location, made, model. One may note that the configuration settings and required driver may be acquired directly from the sensor itself or through the manufacturer's database.

Any subsequent monitoring of the patient John Doe by a practitioner will be enriched with the newly added sensor, either as a primary or secondary sensor. Indeed, the present telemedicine platform is scalable as new sensors may be added and registered for a patient at any time. Nowadays, food intake parameters may be measured using intra body sensors that can measure such parameters based on stomach digestion.

In the present illustrations, reference was made to secondary sensors linked to the first/primary sensor selected by the practitioner. The contextual parameters may be enriched through other means like prerecorded secondary data associated through the mapping rule to the first parameter. The prerecorded secondary data may also be stored in the EMR as it is captured. Such prerecorded data may come from:
periodic (or not) interviews with the patient, prompted through the patient side of the telemedicine platform to collect data related to his uses for eating, coffee or alcohol intake . . .
periodic (or not) measurements of secondary parameters, like food intake, coffee intake, urea . . . using different type of sensors that cannot be triggered with the first sensor when a first parameter is selected by the practitioner for measurement. This may be for instance the data from regular blood checks at a medical test lab that are added to the patient EMR. This may also be results from offline tests from sensors that cannot be controlled on demand through acquisition signals at the time the primary sensors is acquired, like a sensor to measure sugar levels when the patient suffers from diabetes . . .
raisonnement pour défendre 101.
relecture

The present system and controller are deeply rooted into computer technologies as they allow to drive the data acquisition circuits of remoted circuits, whether the primary sensor to measure the selected primary parameter or the subset of secondary sensors, linked to the first one through the mapping rule, so as to measure the selected secondary parameters defining the contextual information useful to the practitioner. Such limitations add significantly more than the simple linking of physiological indications as they solve a network centric problem of acquiring relevant sensor data simultaneously. The combined captured of primary and secondary data allows a practitioner to better understand an output from a telemedicine platform without having himself to perform all the related measurements or repetitive remote captures.

What is claimed is:

1. A data acquisition server for monitoring sensor data on a patient, the server controlling a plurality of sensors, each comprising a data acquisition circuit for measuring data on the patient, the server comprising one or more controllers operable to:
receive selection of a first sensor for capturing a first data on a patient,
retrieve in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
control the data acquisition circuit of the first sensor to capture first data,
control the data acquisition circuit of each secondary sensors from the subset of sensors, to capture secondary data,
retrieve in the sensor database a first rendering rule associated to the first sensor, the first rendering rule defining rules to process the second data prior to rendering,
render on a display controlled by the data acquisition server a user interface comprising the captured first data and the second data based on the first rendering rule.

2. A server as in claim 1, wherein to retrieve of the first mapping rule, the one or more controllers are further operable to:
retrieve in a medical record database a plurality of sensors registered for the patient,
retrieve in the sensor database a predefined mapping rule for the first sensor,
define the first mapping rule by adapting the predefined mapping rule to the plurality of sensors registered for the patient.

3. A server as in claim 1, wherein the first mapping rule comprises for the first sensor and each sensor of the subset of secondary sensors configuration settings to define the acquisition signals for controlling the data acquisition circuit of the sensor.

4. A server as in claim 1, wherein the first mapping rule further comprises prerecorded secondary data linked to the first sensor.

5. A server as in claim 1, wherein the one or more controllers are further operable to:
send a wake up signal to the data acquisition circuit of each sensor.

6. A server as in claim 5, wherein the one or more controllers are further operable to:
display on the user interface a message when at least one of the data acquisition circuits of the sensors does not respond.

7. A server as in claim 1, wherein the first rendering rule defines a combination of the captured secondary data to define a confidence score consolidated over the subset of secondary sensors.

8. A server as in claim 4, wherein the confidence score is rendered in the form of a selectable element on the user interface, the selection of which triggering the rendering of the contribution of each secondary sensor of the subset.

9. A method for monitoring sensor data on a patient using a data acquisition server, the server controlling a plurality of sensors, each comprising a data acquisition circuit for measuring data on the patient, the method comprising the acts of:
receiving selection of a first sensor for capturing a first data on a patient,
retrieving in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
controlling the data acquisition circuit of the first sensor to capture first data,
controlling the data acquisition circuit of each secondary sensors from the subset of sensors, to capture secondary data,
retrieving in the sensor database a first rendering rule associated to the first sensor, the first rendering rule defining rules to process the second data prior to rendering,
rendering on a display controlled by the data acquisition server a user interface comprising the captured first data and the second data based on the first rendering rule.

10. A program product stored on a non-transitory computer-readable storage medium, and executable by a computer in the form of a software agent including at least one software module set up to implement a method for monitoring sensor data on a patient using a plurality of sensors, each comprising a data acquisition circuit for measuring data on the patient, the computer product comprising instructions to:
receive selection of a first sensor for capturing a first data on a patient,
retrieve in a sensor database controlled by the data acquisition server a first mapping rule associated to the first sensor, the first mapping rule defining among the plurality of sensors a subset of secondary sensors linked to the first sensor, a sensor being a secondary sensor to the first sensor when measuring a type of data that characterizes the context of measurement by the first sensor,
control the data acquisition circuit of the first sensor to capture first data,
control the data acquisition circuit of each secondary sensors from the subset of sensors, to capture secondary data,
retrieve in the sensor database a first rendering rule associated to the first sensor, the first rendering rule defining rules to process the second data prior to rendering,
render on a display controlled by the data acquisition server a user interface comprising the captured first data and the second data based on the first rendering rule.
